(19) United States
(12) Patent Application Publication
(10) Pub. No.: US 2024/0269073 A1
(43) Pub. Date: Aug. 15, 2024

(54) CO-AMORPHOUS FORMS FOR USE IN CANCER THERAPY

(71) Applicant: Zerion Pharma APS, Birkerød (DK)

(72) Inventors: Wei Tian, Cleghorn (GB); Donglei Leng, Søborg (DK); Bulut Bulduk, Taastrup (DK); Korbinian Löbmann, Copenhagen N (DK)

(21) Appl. No.: 18/564,552

(22) PCT Filed: Jun. 7, 2022
(86) PCT No.: PCT/EP2022/065413

§ 371 Date: Nov. 27, 2023

(30) Foreign Application Priority Data
Jun. 7, 2021 (EP) 21177986.3

(51) Int. Cl. A61K 9/14; A61K 9/20; A61K 31/454; A61K 31/502; A61K 31/573; A61K 31/58

(52) U.S. Cl. CPC A61K 9/146; A61K 9/2018; A61K 9/2059; A61K 31/454; A61K 31/502; A61K 31/573; A61K 31/58

(57) ABSTRACT

The invention relates to a co-amorphous form of beta-lactoglobulin and a drug substance selected from olaparib and abiraterone acetate, wherein the concentration of the drug substance in the co-amorphous form is from 10% to 90% w/w based on the total weight of the co-amorphous form. I also related to compositions comprising a co-amorphous form, and a dosage regime for the treatment of cancer such as prostate cancer, notably castration-resistant prostate cancer.

### FIELD OF THE INVENTION

The present invention relates to co-amorphous forms for use in cancer therapy. Especially, it relates to co-amorphous forms of a protein and anti-cancer agents such as anti-cancer agents for use in prostate cancer, notably in metastatic castrate-resistant prostate cancer (mCRPC). Typically, the anti-cancer agents are abiraterone acetate, enzalutamide and olaparib and combinations thereof. Typically, the protein is beta-lactoglobulin. The present invention also relates to pharmaceutical compositions comprising the co-amorphous forms, methods for preparing the compositions and dosage regimes for administration of the co-amorphous forms. The invention provides a dosage regime for treatment of cancer comprising oral administration with a significant lower number of compositions than used to date. Thus, the invention also addresses problems relating to non-adherence.

### BACKGROUND OF THE INVENTION

Prostate cancer is cancer of the prostate. The prostate is the gland in the male reproductive system that surrounds the urethra just below the bladder. Most prostate cancers are slow growing. Cancerous cells may spread to other areas of the body, particularly to the bones and lymph nodes. It may initially cause no symptoms. In later stages, symptoms include pain or difficulty in urinating, blood in the urine, or pain in the pelvis or back. Other late symptoms include fatigue, due to low levels of red blood cells.

Globally, it is the second-most common cancer. It is the fifth-leading cause of cancer-related death in men. In 2018, it was diagnosed in 1.2 million and caused over 350,000 deaths. It was the most common cancer in males in more than 80 countries occurring more commonly in the developed world. Detection increased significantly in the 1980s and 1990s in many areas due to increased prostate-specific antigen (PSA) screening.

Many anti-cancer drug substances suffer from low water-solubility or low bioavailability. The present invention is a development of the Applicant's previous findings that the solubility of poorly water-soluble drug substance can be improved by presenting the drug substance in co-amorphous form together with a protein.

Oral delivery is the preferred way of drug administration, since oral formulations are cheap to produce and convenient for the patient. However, oral formulation of crystalline drug substances with poor aqueous solubility is a major challenge for the pharmaceutical industry, since these substances exhibit poor solubility and low dissolution rates, resulting in low bioavailability and poor therapeutic performance.

Amorphous formulations have previously been used for addressing these issues. By converting the crystalline form of a drug into its amorphous counterpart, the solubility and dissolution rate of the drug substance is increased, leading to improved bioavailability and therapeutic efficacy (Hancock et al., Pharm. Res. 17 (2000) pp. 397-404). However, amorphous drug forms are physically unstable and tend to re-crystallize back into the poorly soluble crystalline form during storage (Laitinen et al., Int. J. Pharm. 453 (2013) pp. 65-79). Thus, methods for stabilizing amorphous drug forms are warranted by the pharmaceutical industry. Notably, there is a need in the art for new excipients that can further improve the stability and/or solubility properties of co-amorphous formulations.

Albreht et al. (J. Agric. Food Chem., 2012, 60, 10834-10843) disclose increasing solubility of shikonins using beta-lactoglobulin. The purity of the beta-lactoglobulin used in these experiments was 90%. Furthermore, Albreht et al. did not mention co-amorphous forms of the shikonins with beta-lactoglobulin.

WO 2018/113890 discloses co-amorphous forms of drug substances and various proteins. One of these proteins is beta-lactoglobulin. However, the purity of the beta-lactoglobulin is not specified, and the beta-lactoglobulin used in the examples was from bovine milk with a standard purity of around 90% (from Sigma-Aldrich, Germany). Furthermore, with respect to dissolution enhancement using intrinsic dissolution testing and amorphous physical stability, the highest performing proteins were found to be protein mixtures and in particular whey protein isolate (WPI), which contains approximately 50% to approximately 70% beta-lactoglobulin.

It has been found that beta-lactoglobulin with a higher purity performs better with respect to powder dissolution and physical stability than both WPI and beta-lactoglobulin having the standard purity. Moreover, co-amorphous forms of beta-lactoglobulin with anti-cancer agents have been found to lead to novel dosage regimes where the effective dose of the anti-cancer agents can be markedly reduced leading inter alia to lowering potential side-effects.

Beta-lactoglobulin having higher purity than the standard purity may be prepared according to WO2018/115520.

### DETAILED DESCRIPTION

Patients undergoing cancer treatment are often faced with a high pill burden, i.e. a high number of tablets must be administered during the day in order to obtain the desired effect. In order to reduce the high pill burden and/or to obtain good compliance (i.e. that the patients take the prescribed medication at the prescribed time and in the prescribed amounts), it is advantageous to develop compositions having a high drug load and/or to develop formulations that are smaller in size than the commercially available compositions, but containing the same amount of drug substance or having the same bioavailability but lower amount of the drug than the commercially available compositions.

### Definitions

In the context of the present invention, the term “co-amorphous” refers to a combination of two or more components that form a homogeneous amorphous system where the components are intimately mixed on the molecular level. The “co-amorphous” samples can be prepared by melt and solvent-based approaches, such as spray drying, solvent evaporation, freeze drying, precipitation from supercritical fluids, melt quenching, hot melt extrusion, electrospinning, 2D printing, 3D printing, or by kinetic disordering processes, such as ball milling and cryo-milling. X-ray powder diffraction (XRPD), together with Differential Scanning Calorimetry (DSC), can be used to identify whether the sample is “co-amorphous” after preparation, e.g. by measuring the absence of Bragg peaks and the appearance of a single glass transition temperature.

In the context of the present invention, the term “purity” in connection with the beta-lactoglobulin comprised in the co-amorphous form according to the invention is defined as a percentage (w/w) of beta-lactoglobulin in the total amount of protein comprised in the co-amorphous form. When the co-amorphous form is comprised in a pharmaceutical composition, any additional protein, such as gelatin, that may be included as an excipient in the pharmaceutical formulation does not enter into the calculation of the purity of the beta-lactoglobulin comprised in the co-amorphous form. Furthermore, if an additional protein is included as an excipient in a pharmaceutical composition, said additional protein may give rise to an additional, second glass transition temperature (if amorphous) or melting point (if crystalline) in addition to the glass transition temperature of the co-amorphous form.

In the context of the present invention, the term “drug substance” is intended to refer to a therapeutically active substance. The term “drug substance” refers to a therapeutically active substance. When referring to “a” drug substance in the context of the present invention, it may refer to one or more drug substances.

In the context of the present invention, the term “pill load” or “pill burden” refers to the number of tablets, capsules or other compositions for oral administration, a patient must take on a daily basis. The term “high pill load” or “high pill burden” refers to that more than four oral composition must be taken daily.

In the context of the present invention, the term “compliance” refers to patient compliance, i.e. that the patient takes the prescribed medication at the prescribed time and in the prescribed amounts. It is generally recognized that a patient undergoing therapy requiring a high pill load is at a greater risk for non-compliance compared with patients undergoing therapy with a lower pill load.

In the context of the present invention, the term “mCRPC” refers to metastatic castration-resistant prostate cancer. A patient considered to have mCRPC is a patient with prostate cancer having two conditions identified: i) metastatic prostate cancer, and ii) castration-resistant prostate cancer.

In a first aspect, the invention relates to a co-amorphous form of beta-lactoglobulin and an anti-cancer substance, wherein the concentration of the anti-cancer substance in the co-amorphous form is from 10% to 90% w/w based on the total weight of the co-amorphous form.

The co-amorphous forms and compositions according to the present invention can be used in the treatment of cancer. An important aspect in the treatment of cancer is the risk of non-compliance or non-adherence, i.e. the extent to which patents take medications as prescribed by health care providers.

Cancer medication non-adherence has been shown to lead to decreased survival, higher recurrence/treatment failure rates and health care costs. Cancer in general is a disease that mostly affects older adults. It is estimated that 70% of all incident cases and over 82% of deaths due to cancer occur in persons aged 60 years and over in Canada. Research findings suggest that in the general older population, up to 50% are non-adherent to medication recommendations, which can consequently have serious complications for the health status of an older adult.

A study was carried out to evaluate the adherence to abiraterone or enzalutamide in elderly metastatic castration-resistant prostate cancer by Banna et. al (2020). Fifty-eight patients with a median age of 76 years (56 to 94) were enrolled in the study. A non-adherence wate of 4.8% and 6.2% was observed for the abiraterone and enzalutamide treatment groups respectively. There was a significant correlation between non-adherence and geriatric G8 score (p-0.005). The geriatric G8 score is a screening tool for a full geriatric evaluation of frailty.

Adherence is a multi-dimensional phenomenon, and according to the WHO, is influenced by patient-related factors, therapy-related factors, condition-related factors, health system factors and social economic factors.

Multimorbidity in the older population increases treatment complexity (e.g. conflicting treatments, drug interactions). An increasing number of prescribed medications are associated with decreasing medication adherence in the general older population as well as in older adults who are prescribed oral chemotherapy and/or hormonal therapy. In addition, many of the anti-cancer oral medications suffer from poor solubility and bioavailability. Therefore, the patients are presented with multiple tablets, often very large for one disease, which are then multiplied for other morbidities. Dysphagia refers to difficulties in swallowing. Elderly patients are inherently predisposed to dysphagia predominately because of comorbid health conditions.

The prevalence of dysphagia in the Midwestern US population was reported to be 6% to 9%, its prevalence in community-dwelling persons over age 50 years is estimated to be between 15% and 22%. The prevalence of dysphagia is even higher in those residing in assisted living facilities and nursing homes, where up to 40% to 60% of residents are reported to have feeding difficulties (Aslam & Vaezi, 2013). Dysphagia would not only negatively the patient's quality of life as they have to overcome the swallowing difficulty to medicate on a daily basis, it can also lead to medication non-adherence.

Therefore, there is a need to reduce the pill burden for the elderly patients, and the elderly cancer patients in particular to improve their quality of life and to enhance their adherence to the prescribed therapeutic regimen.

### Prostate Cancer

Prostate cancer is the most common cancer amongst males in the UK, accounting for 26% of all new cancer cases in this population (2017 data). The main function of the prostate is to produce fluids that form part of semen. Advance prostate cancer means the cancer has spread from the prostate to other parts of the body (metastatic prostate cancer). It most commonly spreads to lymph nodes in other parts of the body and to the bones. It can also spread to other organs.

Prostate cancer normally need testosterone to grow. Prostate cancer that has spread to other parts of the body and which keeps growing even when the amount of testosterone in the body is reduced to a very low level (via testosterone suppression therapy) is denoted mCRPC. Prostate-specific membrane antigen (PSMA), a transmembrane protein, is expressed by virtually all prostate cancers, and its expression is further increased in poorly differentiated, metastatic, and hormone-refractory carcinomas.

Prostate cancer is more common in black Caribbean and black African men than in white men and is less common in Asian men. Around 35% of the men diagnosed with prostate cancer in the UK each year are aged 75 years and over. Additional factors which increase the risk of developing prostate cancer include having a family history of the condition, and lifestyle factors such as consuming a lot of red meat and foods that are high in fat.

Advanced prostate cancer can cause symptoms, such as fatigue (extreme tiredness), bone pain, and problems urinating. The symptoms also depend on where the cancer has spread to. Prostate cancer is a significant cause of morbidity and mortality in men, especially in those over age of 75 years and impacts on their daily lives, particularly physical and emotional health, relationship and social life.

The current treatment options are i) abiraterone, which is recommended together with prednisone or prednisolone, ii) enzalutamide, which is recommended for treating metastatic hormone-relapsed prostate cancer, and iii) docetaxel, which is recommended for treating hormone-refractory prostate cancer only if the patients' Karnofsky performance-score is 60% or more.

The current dose of abiraterone acetate is 1000 mg once daily given as 4×250 mg. Moreover, prednisolone is given twice daily. The tablets provided as Zytiga from Johnson and Johnson are very large, oval-shaped, film coated tablets, and the 500 mg tablet is 20 mm long and 10 mm wide, whereas the 250 mg tablet is 15.9 mm long×9.5 mm wide.

### Co-Amorphous Forms

In the context of the present invention, the term “co-amorphous” refers to a combination of two or more components, such as three or more components, that form a homogeneous amorphous system where the components are intimately mixed in a single phase or two phases. X-ray powder diffraction (XRPD), together with Differential Scanning Calorimetry (DSC), can be used to identify whether the sample is “co-amorphous” after preparation, e.g. by measuring the absence of Bragg peaks and the appearance of one or two glass transition temperature(s), i.e. at least one less T_{g} than would be observed if no mixing on the molecular level took place (e.g. one T_{g} in a two-component system, one or two T_{g}'s in a three-component system).

The present invention concerns a co-amorphous form of a drug substance and a protein. The drug substance is an anti-cancer substance, and the protein is beta-lactoglobulin, wherein the purity of the beta-lactoglobulin is at least 92% (w/w) of the total amount of protein comprised in the co-amorphous form. Without being bound by a particular theory, it has been found that the purity of the beta-lactoglobulin contributes positively towards a higher solubility and/or stability of the drug substance. Accordingly, in one embodiment of the present invention, the purity of the beta-lactoglobulin in the co-amorphous form of the invention is at least 94% (w/w) of the total amount of protein comprised in the co-amorphous form. In another embodiment of the present invention, the purity of the beta-lactoglobulin in the co-amorphous form of the invention is at least 95% (w/w) of the total amount of protein comprised in the co-amorphous form. In still another embodiment of the present invention, the purity of the beta-lactoglobulin in the co-amorphous form of the invention is at least 96% (w/w) of the total amount of protein comprised in the co-amorphous form. In yet another embodiment, the purity of the beta-lactoglobulin in the co-amorphous form of the invention is at least 97% (w/w) of the total amount of protein comprised in the co-amorphous form. In a further embodiment of the present invention, the purity of the beta-lactoglobulin in the co-amorphous form of the invention is at least 98% (w/w) of the total amount of protein comprised in the co-amorphous form.

The co-amorphous form of the invention may contain from 1 to 99% (w/w) of the drug substance, such as from 5 to 95% (w/w) of the drug substance. In embodiments, the co-amorphous form comprises from 10 to 90% (w/w) of the drug substance and from 10 to 90% (w/w) of the beta-lactoglobulin, or the co-amorphous form comprises from 20 to 90% (w/w) of the drug substance and from 10 to 80% (w/w) of the beta-lactoglobulin. The co-amorphous form may also comprise from 30 to 85% (w/w) of the drug substance and from 15 to 70% (w/w) of the beta-lactoglobulin, from 50 to 85% (w/w) of the drug substance and from 15 to 50% (w/w) of the beta-lactoglobulin, from 55 to 75% (w/w) of the drug substance and from 25 to 45% (w/w) of the beta-lactoglobulin. In yet another embodiment, the co-amorphous form comprises from 30% to 60% (w/w) of the drug substance and from 40% to 70% (w/w) of the beta-lactoglobulin. As seen from the examples herein suitable co-amorphous forms are achieved containing 30% (w/w) drug substance and 70% (w/w) beta-lactoglobulin 40% (w/w) drug substance and 60% (w/w) beta-lactoglobulin, 50% (w/w) drug substance and 50% (w/w) beta-lactoglobulin.

When a high drug load is of interest, the co-amorphous form may comprise more than 50% (w/w) of the drug substance such as 60% (w/w) of the drug substance and 40% (w/w) of the beta-lactoglobulin, or 70% (w/w) of the drug substance and 30% (w/w) of the beta-lactoglobulin.

In a further aspect, the present invention concerns the use of a beta-lactoglobulin having a purity of at least 92% (w/w) for preparing a co-amorphous form with a drug substance.

The co-amorphous forms may be prepared according to the present examples or according to the general methods disclosed in WO 2018/113890. Accordingly, in one aspect, the present invention concerns a method of preparing a co-amorphous form of the invention, said method selected from subjecting the drug substance and beta-lactoglobulin together to spray drying, solvent evaporation, freeze drying, precipitation from supercritical fluids, melt quenching, hot melt extrusion, electrospinning, 2D printing, 3D printing, and any milling process, such as ball milling and cryo-milling.

As mentioned above, there is a need for reducing the pill burden in cancer treatment. The present inventors have found that it is possible to reduce the pill burden by use of compositions that comprises the drug substance in co-amorphous form with a beta-lactoglobulin. Firstly, it is possible to obtain such co-amorphous forms having a high concentration of the drug substance and, secondly, it seems as if the co-amorphous forms have technical properties suitable for preparation of solid dosage forms, which makes it possible to reduce the amount of pharmaceutically acceptable excipients that is used. Suitable properties include good flowability, good compressibility etc.

Preferably, the anti-cancer drug substance is selected from olaparib, abiraterone acetate and enzalutamide and combinations thereof, notably in further combination with one or more corticosteroid(s).

The invention provides a co-amorphous form of beta-lactoglobulin and a drug substance selected from olaparib, enzalutamide and abiraterone acetate, wherein the concentration of the drug substance in the co-amorphous form is from 10% to 90% w/w based on the total weight of the co-amorphous form.

In those cases where the drug substance is olaparib, the concentration of olaparib in the co-amorphous form is typically from 20% to 90% w/w such as from 40% to 75% w/w, based on the total weight of the co-amorphous form.

In those cases where the drug substance is abiraterone acetate or enzalutamide, the concentration of abiraterone acetate or enzalutamide in the co-amorphous form is from 10% to 70% w/w such as from 20% to 60% w/w, based on the total weight of the co-amorphous form.

The purity of beta-lactoglobulin is typically 92% or more. As described herein the inventors have observed that the purity of beta-lactoglobulin in the co-amorphous form may have influence on the water-solubility/dissolution and stability of the co-amorphous form. Thus, it is contemplated that a purity of 92% or more gives improved properties of the co-amorphous form compared with a co-amorphous form wherein beta-lactoglobulin has a lower degree of purity.

As mentioned herein before cancer treatment is often accompanied with administration of a corticosteroid. In embodiments of the invention, the co-amorphous from may be mixed with corticosteroid, or the co-amorphous form may be a co-amorphous form of beta-lactoglobulin, a corticosteroid and an anti-cancer drug substance.

A co-amorphous form according to the present invention can be used in medicine such as in the treatment of cancer. Notably a co-amorphous form according to the invention can be used in the treatment of prostate cancer such as castration-resistant prostate cancer (CRPC).

### Beta-Factoglobulin (BLG)

Beta-lactoglobulin is the major whey protein in the milk of ruminants and many other mammals. Whey refers to the liquid supernatant that is left after the casein of milk has been precipitated and removed (during cheese production). However, beta-lactoglobulin may also be isolated directly from milk. Bovine beta-lactoglobulin is a protein of 162 amino acids, having a molecular weight of approximately 18.4 kDa. Under physiological conditions, the protein is predominantly dimeric (in an open form) while it dissociates to the monomeric state (closed conformation) at pH below 3. The pH is also important for the crystallization of bovine beta-lactoglobulin that may form different lattices depending on the pH. Several genetic variants of beta-lactoglobulin have been identified, the main bovine ones termed A and B. In one embodiment of the present invention, beta-lactoglobulin is beta-lactoglobulin obtained from mammalian species, such as cow, sheep or goat, in its native and/or glycosylated form and includes the genetic variants. It is contemplated as part of the present invention that also modifications including additions, deletions, substitutions of amino acids in the protein of the naturally occurring forms and variants thereof, or recombinant forms of beta-lactoglobulin are useful in the present invention. In a further embodiment, the beta-lactoglobulin is bovine beta-lactoglobulin.

### Drug Substances

The drug substance of the co-amorphous form is an anti-cancer drug substance. Preferably, the drug substance is for the treatment of prostate cancer, notably castration-resistant prostate cancer (CRPC). Available treatment options today are androgen-receptor-targeted therapies (abiraterone acetate or enzalutamide), chemotherapy (docetaxel, cabazitaxel), therapy with inhibitors of the nuclear enzyme poly-(ADP-ribose) polymerase (PARP), or combinations thereof. Often the therapy is combined with administration of a corticosteroid such as prednisone, prednisolone, dexamethasone or the like.

### Olaparib

Olaparib is a member of the class of N-acylpiperazines obtained by formal condensation of the carboxy group of 2-fluoro-5-[(4-oxo-3,4-dihydrophthalazin-1-yl)methyl]-benzoic acid with the free amino group of N-(cyclopropylcarbonyl)piperazine; used to treat advanced cancer. It has a role as an antineoplastic agent, an EC 2.4.2.30 (NAD (+) ADP-ribosyltransferase) inhibitor and an apoptosis inducer. It is a N-acylpiperazine, a member of cyclopropanes, a member of monofluorobenzenes and a member of phthalazines.

The chemical structure is shown below. Olaparib is a crystalline solid. The water-solubility is very poor and pH independent (about 0.06 mg/mL). It is classified as Class 4 according to the Biopharmaceutical Classification System.

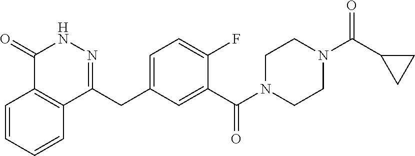 [SMILES: O=C(c1cc(Cc2n[nH]c(=O)c3ccccc23)ccc1F)N1CCN(C(=O)C2CC2)CC1]

Olaparib is a small molecule inhibitor of the nuclear enzyme poly-(ADP-ribose) polymerase (PARP) with potential chemosensitizing, radiosensitizing, and antineoplastic activities. Olaparib selectively binds to and inhibits PARP, inhibiting PARP-mediated repair of single strand DNA breaks; PARP inhibition may enhance the cytotoxicity of DNA-damaging agents and may reverse tumor cell chemoresistance and radioresistance. PARP catalyzes post-translational ADP-ribosylation of nuclear proteins and can be activated by single-stranded DNA breaks. Olaparib therapy is associated with a low rate of transient elevations in serum aminotransferase during therapy and has not been linked to instances of clinically apparent liver injury.

Olaparib is sold under the name Lynparza by AstraZeneca Pharmaceuticals in the form of tablets for oral administration having a strength of 100 mg and 150 mg tablets, or 50 mg capsules. Following oral administration, the absorption of olaparib is very rapid and can reach a peak concentration ranging between 4.7 and 9.1 mcg/ml after 1-3 hours. The reported AUC of olaparib after a dose of 200 mg is of 25.8 mcg·h/L and this AUC can be increased by 26% with constant administration. The consumption of a high-fat diet with olaparib can decrease the tmax but does not have an effect in the peak concentration.

From the administered dose, approximately 86% of the administered dose is recovered after 7 days from which 44% is found in the urine and 42% is obtained in feces.

Olaparib is extensively metabolized in the liver by the action of CYP3A isoenzymes. From the administered dose, the unchanged form of olaparib accounted for 70% of the circulating dose and it was considered the major component in urine and feces. The metabolic pathway of olaparib is mainly attributable to oxidation reactions with subsequent glucuronide and sulfate conjugation. However, the over 20 metabolites found in plasma, urine, and feces represented a minor portion of the administered dose. The major circulating metabolites were represented by the mono-oxygenated form and the piperazin-3-ol form.

Olaparib is an inhibitor of poly (ADP-ribose) polymerase (PARP) enzymes, including PARP1, PARP2, and PARP3. PARP enzymes are involved in normal cellular homeostasis, such as DNA transcription, cell cycle regulation, and DNA repair. Olaparib has been shown to inhibit growth of selected tumor cell lines in vitro and decrease tumor growth in mouse xenograft models of human cancer both as monotherapy or following platinum-based chemotherapy. Increased cytotoxicity and anti-tumor activity following treatment with olaparib were noted in cell lines and mouse tumor models with deficiencies in BRCA [FDA Label]. In vitro studies have shown that olaparib-induced cytotoxicity may involve inhibition of PARP enzymatic activity and increased formation of PARP-DNA complex, resulting in disruption of cellular homeostasis and cell death

Side effects include gastrointestinal effects such as nausea, vomiting, and loss of appetite; fatigue; muscle and joint pain; and low blood counts such as anemia, with occasional leukemia.

Recently FDA approved the drug substance Olaparib (Lynparza) (and rucaparib, Rubraca) for use in men whose prostate cancer has spread, or metastasized, and whose disease has stopped responding to standard hormone treatments, often called castration-resistant disease. To receive either drug, men must also have specific genetic alterations that prevent their cells from repairing damage to their DNA.

Many treatments of metastatic prostate cancer are centered around therapies that block the ability of hormones to fuel the cancer's growth and spread. But olaparib and rucaparib, which are taken as pills, work differently. They block the activity of a protein known as PARP (poly-ADP-Ribose-Polymerases), which helps cells mend specific types of damage to DNA.

Studies have shown that 20%-30% of men with metastatic prostate cancer have genetic alterations that impair cells' DNA repair mechanisms.

Over the past decade, olaparib and rucaparib have become important treatments for women with ovarian and breast cancer, in whom genetic alterations that affect DNA repair processes are common. Among the most frequent such alterations are those in the BRCA1 and BRCA2 genes.

It is no accident that researchers have identified people who have alterations in BRCA genes as ideal candidates for treatment with PARP inhibitors.

BRCA proteins and some PARP proteins are both integral components of cells' response to DNA damage. If that response is already dysfunctional because of BRCA1 or BRCA2 mutations, then researchers reasoned that blocking the activity of PARP proteins could further hamper any chance of repair-akin to punching a hole in a tire that already has a slow leak. If the cancer cells cannot fix the DNA damage, they will die.

Prostate cancer emerged as another strong candidate for PARP inhibitors after studies suggested that alterations in BRCA1 and BRCA2, as well as other genes involved in a cell's ability to respond to DNA damage, may be present in about 25% of men with the disease. Other studies linked these genetic changes to an increased risk of prostate cancer, as well as more aggressive diseases.

Olaparib has been subject to a large clinical trial called PROFOUND.

The trial enrolled men with mutations in DNA repair genes and divided them into two cohorts. Cohort A included men with alterations in the BRCA1, BRCA2, or ATM genes, each of which plays an important role in DNA repair. Cohort B included men who had alterations in a group of 12 other genes that have some involvement in repairing DNA.

All of the men in the trial had cancer that had worsened despite treatment with either abiraterone (Zytiga) or enzalutamide (Xtandi), which work in different ways to block hormones in prostate cancer cells.

The 387 men in the trial were randomly assigned to either the treatment group, which received olaparib, or the control group, which received either abiraterone or enzalutamide (as selected by each patient's oncologist).

In cohort A, men treated with olaparib lived more than twice as long without evidence of their cancer getting worse than men treated with abiraterone or enzalutamide: a median of 7.4 months versus 3.6 months. The treatment group in cohort A also lived longer overall, with olaparib improving survival by more than 4 months (19.1 months versus 14.7 months).

In addition, men treated with olaparib were far more likely to see their tumors shrink (a tumor response) than men treated with one of the other two drugs (33% versus 2%).

FDA's approval covers the use of the drug in men with alterations in any of the DNA repair genes analyzed in the trial. But Dr. Sartor, who also was an investigator on the trial, noted that men with alterations in BRCA2 seemed to respond best to the treatment, experiencing the largest improvement in progression-free survival. The following information is given regarding the commercially available product Lynparza®:

Lynparza is a poly (ADP-ribose) polymerase (PARP) inhibitor indicated:

Ovarian cancer
for the maintenance treatment of adult patients with deleterious or suspected deleterious germline or somatic BRCA-mutated advanced epithelial ovarian, fallopian tube or primary peritoneal cancer who are in complete or partial response to first-line platinum-based chemotherapy. Select patients for therapy based on an FDA-approved companion diagnostic for Lynparza.
in combination with bevacizumab for the maintenance treatment of adult patients with advanced epithelial ovarian, fallopian tube or primary peritoneal cancer who are in complete or partial response to first-line platinum-based chemotherapy and whose cancer is associated with homologous recombination deficiency (HRD)-positive status defined by either:
a deleterious or suspected deleterious BRCA mutation, and/or
genomic instability. Select patients for therapy based on an FDA-approved companion diagnostic for Lynparza.
for the maintenance treatment of adult patients with recurrent epithelial ovarian, fallopian tube or primary peritoneal cancer, who are in complete or partial response to platinum-based chemotherapy.
for the treatment of adult patients with deleterious or suspected deleterious germline BRCA-mutated (gBRCAm) advanced ovarian cancer who have been treated with three or more prior lines of chemotherapy. Select patients for therapy based on an FDA-approved companion diagnostic for Lynparza.

Breast cancer
for the treatment of adult patients with deleterious or suspected deleterious gBRCAm, HER2-negative metastatic breast cancer who have been treated with chemotherapy in the neoadjuvant, adjuvant or metastatic setting. Patients with hormone receptor (HR)-positive breast cancer should have been treated with a prior endocrine therapy or be considered inappropriate for endocrine therapy. Select patients for therapy based on an FDA-approved companion diagnostic for Lynparza.

Pancreatic cancer
for the maintenance treatment of adult patients with deleterious or suspected deleterious gBRCAm metastatic pancreatic adenocarcinoma whose disease has not progressed on at least 16 weeks of a first-line platinum-based chemotherapy regimen. Select patients for therapy based on an FDA-approved companion diagnostic for Lynparza.

Prostate cancer
for the treatment of adult patients with deleterious or suspected deleterious germline or somatic homologous recombination repair (HRR) gene-mutated metastatic castration-resistant prostate cancer

Lynparza® has the following size:

$$Lynparza 100 mg : length \times width \times height : 14.7 mm \times 7.4 mm \times 4.7 mm$$

$$Lynparza 150 mg : length \times width \times height : 14.7 mm \times 7.4 mm \times 6.6 mm$$

### Abiraterone and Abiraterone Acetate

Abiraterone acetate ([(3S,8R,9S,10R,13S,14S)-10,13-dimethyl-17-pyridin-3-yl-2,3,4,7,8,9,11,12,14,15-decahydro-1H-cyclopenta[a]phenanthren-3-yl] acetate) is a sterol ester obtained by formal condensation of the 3-hydroxy group of abiraterone with the carboxy group of acetic acid. A prodrug that is converted in vivo into abiraterone. Used for treatment of metastatic castrate-resistant prostate cancer. In the present context, both abiraterone and abiraterone acetate can be used as well as other prodrugs of abiraterone.

Abiraterone acetate is an orally active acetate ester form of the steroidal compound abiraterone with antiandrogen activity. Abiraterone inhibits the enzymatic activity of steroid 17alpha-monooxygenase (17alpha-hydrolase/C17,20 lyase complex), a member of the cytochrome p450 family that catalyzes the 17alpha-hydroxylation of steroid intermediates involved in testosterone synthesis. Administration of this agent may suppress testosterone production by both the testes and the adrenals to castrate-range levels.

It is a sterol ester and a member of pyridines. Its structure is

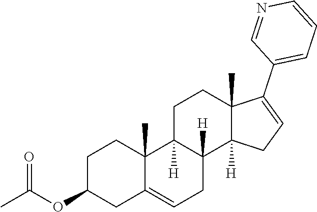

It has poor solubility in water, i.e. less than 1 mg/mL. It is a white to off-white, non-hygroscopic, crystalline powder.

Abiraterone acetate is sold under the brand name Zytiga among others, and is a medication used to treat prostate cancer. Specifically, it is used together with a corticosteroid for metastatic castration-resistant prostate cancer (mCRPC) and metastatic castration-sensitive prostate cancer (mCSPC). It should either be used following removal of the testicles or along with a gonadotropin-releasing hormone (GnRH) analog. It is taken by mouth and administered as tablets having a strength of 125 mg, 250 or 500 mg.

Common side effects include tiredness, vomiting, headache, joint pain, high blood pressure, swelling, low blood potassium, high blood sugar, hot flashes, diarrhea, and cough. Other severe side effects may include liver failure and adrenocortical insufficiency. In males whose partners can become pregnant, birth control is recommended. Supplied as abiraterone acetate it is converted in the body to abiraterone. Abiraterone acetate works by suppressing the production of androgens—specifically it inhibits CYP17A1—and thereby decreases the production of testosterone. In doing so, it prevents the effects of these hormones in prostate cancer.

Abiraterone acetate was described in 1995 and approved for medical use in the United States and Europe in 2011. It is on the World Health Organization's List of Essential Medicines, the safest and most effective medicines needed in a health system.

In 2017, abiraterone acetate emerged as the first androgen-signaling inhibitor (ASI) to receive FDA approval in mCSPC. The placebo-controlled, Phase 3 LATITUDE trial randomly assigned 1199 patients with high-risk mCSPC, defined as a Gleason score of 8 or more, at least three bone lesions or presence of measurable visceral metastasis (at least 2 of 3 criteria met), to Abiraterone acetate 1000 mg daily with prednisone 5 mg twice daily and a GnRH agonist/antagonist or GnRH agonist/antagonist alone. Markedly improved mOS was observed in the Abiraterone acetate group—53.3 months (95% CI, 48.2—not yet reached) Abiraterone acetate arm vs 36.5 months (95% CI, 33.5-40.0) placebo arm (HR, 0.62; P<0.001). mPFS was reported at almost 3 years—33.0 months in the Abiraterone acetate group vs 14.8 months in the placebo group (HR, 0.47; 95% CI, 0.39-0.55; P<0.001). These findings were later confirmed by STAMPEDE arm G in which the Abiraterone acetate arm demonstrated a 3-year survival rate of 83% compared to 76% in the ADT-alone group (HR, 0.63; P<0.001). As in the STAMPEDE arm C analysis (docetaxel), high risk localized and lymph node positive, non-nonmetastatic patients were included, as well.

The following products are commercially available.

ONSA is a CYP17 inhibitor indicated in combination with methylprednisolone for the treatment of patients with metastatic castration-resistant prostate cancer (CRPC).

ZYTIGA is a CYP17 inhibitor indicated in combination with prednisone for the treatment of patients with metastatic castration-resistant prostate cancer (CRPC).

The size of Zytiga® 500 mg: length×width×height: 20 mm×10 mm×7.5 mm

### Enzalutamide

Enzalutamide is a non-steroidal antiandrogen (NSAA) drug substance which is used in the treatment of prostate cancer.

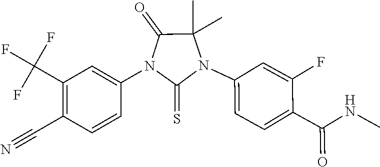 [SMILES: CNC(=O)c1ccc(N2C(=S)N(c3ccc(C#N)c(C(F)(F)F)c3)C(=O)C2(C)C)cc1F]

Enzalutamide (4-[3-[4-cyano-3-(trifluoromethyl)phenyl]-5,5-dimethyl-4-oxo-2-sulfanylideneimidazolidin-1-yl]-2-fluoro-N-methylbenzamide) is a benzamide obtained by formal condensation of the carboxy group of 4-{3-[4-cyano-3-(trifluoromethyl)phenyl]-5,5-dimethyl-4-oxo-2-thioxoimidazolidin-1-yl}-2-fluorobenzoic acid with methylamine. Used for the treatment of metastatic castration-resistant prostate cancer. It has a role as an antineoplastic agent and an androgen antagonist. It is a member of benzamides, an imidazolidinone, a thiocarbonyl compound, a nitrile, a member of (trifluoromethyl)benzenes and a member of monofluorobenzenes. It is practically insoluble in water, i.e. it has a water solubility of less than 1 mg/mL. It is sold under the trade name Xtandi by Astellas Pharma US Inc. It is available in the form of capsules or tablets for oral administration containing 40 mg of enzalutamide. It may be used in doses of 80 mg and gradually increasing the dose to 160 mg.

Enzalutamide is hepatically metabolized, primarily by CYP2C8 and CYP3A4. The enzyme that converts enzalutamide to its active metabolite, N-desmethyl enzalutamide, is CYP2C8. The activity of N-desmethyl-enzalutamide is similar to that of the parent compound.

The mean terminal half-life (t½) for enzalutamide in patients after a single oral dose is 5.8 days (range 2.8 to 10.2 days). Following a single 160 mg oral dose of enzalutamide in healthy volunteers, the mean terminal t½ for N-desmethyl enzalutamide is approximately 7.8 to 8.6 days.

Enzalutamide is 97% to 98% bound to plasma proteins, primarily albumin. N-desmethyl enzalutamide is 95% bound to plasma proteins.

Enzalutamide is a competitive androgen receptor inhibitor that effects multiple stages of the signaling pathway. It is able to inhibit androgen binding to its receptor, androgen receptor nuclear translocation, and subsequent interaction with DNA. As a result, proliferation of prostate cancer cells decreases which ultimately leads to apoptosis and decreased tumor volume.

It is indicated for use in conjunction with castration in the treatment of metastatic castration-resistant prostate cancer (mCRPC), non-metastatic castration-resistant prostate cancer and metastatic castration-sensitive prostate cancer (mCSPC). It is administered orally.

Side effect of enzalutamide when added to castration include asthenia (abnormal physical weakness or lack of energy), back pain, arthralgia (joint pain), hot flush, peripheral edema, headache, upper respiratory infection, dizziness, insomnia, lower respiratory infection, anxiety and hypertension. It may cause seizures and it has a high potential for drug interactions. As an antiandrogen it acts as an antagonist of the androgen receptor, the biological targets of androgens like testosterone and dihydrotestosterone, thereby preventing the effects of these hormones in the prostate gland and elsewhere in the body.

Enzalutamide was introduced for the treatment of prostate cancer in 2012.

The development of enzalutamide occurred in the context of mCRPC, specifically aiming to overcome resistance to androgen deprivation (ADT) monotherapy. Enzalutamide demonstrates high affinity for androgen receptor (AR), competitively overcoming well-described mechanisms of castration resistance including AR overexpression, extragonadal testosterone synthesis, and acquired sensitivity to non-androgen ligands. Enzalutamide also inhibits AR nuclear translocation and binding with elements of transcription. The therapeutic advantage of early enzalutamide use in CSPC mechanistically remains poorly understood. One possibility is that more complete inhibition of AR signaling delays the emergence of castration resistance mechanisms. Molecular changes in response to ADT monotherapy may also prime prostate cancer cells to respond less favorably to enzalutamide in the castration-resistant setting (ie, AR splice variants, mutations in the ligand-binding domain, or upregulation of non-AR mediated signaling pathways).

The following product is commercially available:

XTANDI is an androgen receptor inhibitor indicated for the treatment of patients with:
castration-resistant prostate cancer.
metastatic castration-sensitive prostate cancer.

### Combination of Abiraterone Acetate (or Abiraterone) and Olaparib

Recent clinical studies have revealed advantages in the treatment with prostate cancer by use of a combination of abiraterone acetate and olaparib.

Olaparib in combination with abiraterone acetate is in clinical development for patients with metastatic castration-resistant prostate cancer. The cancer is called mCRPC when the cancer cells have spread to other parts of the body like bones, lymph nodes outside the pelvis or rarely to the liver or lungs. It is not possible to cure mCRPC but it is possible to keep it under control. In the clinical study, olaparib is administered orally in tablet form and can lead to cancer cell death by blocking DNA repair by an enzyme PARP. By blocking PARP enzymes, the damaged DNA in cancer cells cannot be repaired, and the cells will die. Abiraterone works by stopping the body making testosterone which subsequently stops the cancer growing.

In the current clinical study (phase III clinical trial, PROpel, NCT03732820), participants receive olaparib 300 mg (2×150 mg) orally twice daily plus abiraterone acetate 1000 mg orally once daily. Patients also receive prednisone or prednisolone 5 mg twice daily. The primary completion date of the study was April 2021.

### Combination of Enzalutamide and Olaparib

Abiraterone acetate and enzalutamide have the same mechanism of action and therefore combinations of enzalutamide and olaparib are also within the scope of the present invention.

Combination with Corticosteroid

Often a dosage regime for treatment of cancer such as prostate cancer involves the use of a corticosteroid such as prednisone or prednisolone. The dosage of the corticosteroid depends inter alia on the specific drug substance, the age and condition of the patient and of the disease to be treated.

The term “hydrocortisone equivalents” is used herein to define the amount in mg of a specific glucocorticoid that corresponds to 1 mg of hydrocortisone for the purpose of glucocorticoid therapy as generally understood by medical practitioners. The term is based on the fact that the individual glucocorticoids have different potency and in order to achieve a desired therapeutic effect different doses of the individual glucocorticoids are required. Equivalent doses of the glucocorticoids can be calculated based on the following table.

| | | Hydrocortisone equivalent |
|---|---|---|
| | | (1 mg of the glucocorticoid |
| | Equivalent | corresponds to the listed |
| Glucocorticoid | amount (mg) | amount in mg of hydrocortisone) |
| Cortisone acetate | 25 | 0.8 |
| Hydrocortisone | 20 | 1 |
| Prednisolone | 5 | 4 |
| Prednisone | 5 | 4 |
| Methylprednisolone | 4 | 5 |
| Triamcinolone | 4 | 5 |
| Paramethasone | 2 | 10 |
| Betamethasone | 0.75 | 26.66 |
| Dexamethasone | 0.75 | 26.66 |
| Fludrocortisone | 0.05 | 400 |

In the treatment of castration-resistant prostate cancer, a dose corresponding to 5 mg of prednisone or 5 mg of prednisolone is normally administered together with the anti-cancer drug substance (e.g. olaparib and/or abiraterone acetate).

### Pharmaceutical Compositions

The co-amorphous forms of the invention may be included in a pharmaceutical composition. Hence, in one aspect of the invention, it concerns a pharmaceutical composition comprising a co-amorphous form according to the invention and at least one pharmaceutically acceptable carrier or excipient.

A composition of the present invention typically contains from 5% to 100% w/w such as from 5% to 95% w/w of the co-amorphous form, based on the total weight of the composition and the type of dosage form.

When the drug substance is olaparib, the concentration of the co-amorphous form is typically from 5% to 95% w/w such as from 10% to 80% w/w, from 20% to 70% w/w, from 30% to 70% w/w or from 40% to 60% w/w of the total weight of the composition.

When the drug substance is abiraterone acetate or enzalutamide, the concentration of the co-amorphous form is typically from 5% to 95% w/w such as from 10% to 90% w/w, from 20% to 85% w/w, from 30% to 85% w/w or from 40% to 85% w/w of the total weight of the composition.

The compositions may further comprise niraparib, either as part of the co-amorphous form or separately in the composition.

The co-amorphous forms of the invention are preferably formulated with a pharmaceutically acceptable carrier or excipient. A pharmaceutically acceptable carrier or excipient is an inert carrier or excipient suitable for each administration method and can be formulated into conventional pharmaceutical preparation (tablets, granules, capsules, powder, solution, suspension, emulsion, injection, infusion, etc.). As such a carrier or excipient there may be mentioned, for example, a binder, a lubricant, a disintegrant and the like, which are pharmaceutically acceptable. When they are used as an injection suspension or an infusion suspension, they can be formulated by using distilled water for injection, physiological saline, an aqueous glucose solution.

The administration method of the pharmaceutical compositions of the present invention is not particularly limited, and a usual oral or parenteral administration method (intravenous, intramuscular, subcutaneous, percutaneous, intranasal, transmucosal, enteral, etc.) can be applied. In one embodiment, the pharmaceutical composition is in a form suitable for oral or nasal administration, such as a solid formulation, powder, tablets, capsule, granules, sachets, reconstitutable powders, powders, dry powder inhalers and chewables or oral solutions/suspensions. Preferably, the composition is a solid dosage form.

Preferably, the composition of the invention is for oral administration. The composition is in the form of a dosage form such as tablets, capsules, pellets, sachets etc. As appears from the Examples it is possible to prepare a solid dosage form such as tablets with a high load of drug substance (from about 35% to 80% w/w, preferably from 45% to 80% w/w co-amorphous form) and with at the most 55% w/w of pharmaceutically acceptable excipients.

A composition of the present invention may further comprise one or more corticosteroids.

A composition of the present invention can be used in medicine such as in the treatment of cancer including prostate cancer such as castration-resistant prostate cancer.

It is contemplated that the co-amorphous form has good tableting properties and therefore it is not necessary with a high amount of pharmaceutically acceptable excipients. As an example, solid dosage forms can be prepared using
Co-amorphous form 45% to 80% w/w such as from 50% to 75% w/w
Direct compressible starch (e.g. StarTab®) 10% to 55% w/w such as from 10% to 40% w/w
Diluent or plasticizer (e.g. sorbitol) 5% to 25% w/w such as from 5% to 15% w/w.

Optional ingredients are
Superdisintegrant (e.g. Explotab®) 2% to 7% such as from 5 to 6.5%
Lubricant (e.g. sodium stearyl fumarate) 0.01% to 1.5% w/w such as from 0.1% to 1% w/w
Surfactant (e.g. sodium lauryl sulphate) 2.5% to 10% w/w such as 2.5% to 5% w/w.

### Dosage Regimes

The present invention also relates to a dosage regime for the treatment of prostate cancer, notably castration-resistant prostate cancer.

A dosage regime of the present invention comprises daily administering an initial dose of 300 mg olaparib and 1000 mg of abiraterone acetate followed by a dose of 300 mg olaparib 12 hours after administration of the initial dose for the treatment of prostate cancer such as castration-resistant prostate cancer, wherein the dose of olaparib is contained in a co-amorphous form of the invention or in one or more compositions of the invention and the dose of abiraterone acetate is contained in a co-amorphous form of the invention or in one or more compositions of the invention.

It is contemplated that the dose of abiraterone acetate may be markedly reduced due to better solubility and bioavailability of abiraterone acetate in the co-amorphous form. Accordingly, the present invention also provides a dosage regime comprising daily administering an initial dose of 300 mg or 150 mg olaparib and below 1000 mg, such as 500 mg, 250 mg, 200 mg or 150 mg, abiraterone acetate followed by a dose of 300 mg or 150 mg olaparib 12 hours after administration of the initial dose for the treatment of prostate cancer such as castration-resistant prostate cancer. Olaparib and/or abiraterone acetate is typically in the form of the co-amorphous forms with beta-lactoglobulin.

The dosage regime may further comprise administering an initial dose of a corticosteroid such as e.g. 5 mg prednisone or prednisolone followed by a second dose of the corticosteroid such as e.g. 5 mg prednisone or prednisolone 12 hours after administration of the initial dose. The initial dose of olaparib and abiraterone acetate is administered at the same time as the initial dose of prednisone or prednisolone.

The dose of corticosteroid may be provided as a separate composition or it is included in the composition comprising olaparib, either admixed with the co-amorphous form of beta-lactoglobulin and olaparib or in the form of a co-amorphous form of beta-lactoglobulin, olaparib and corticosteroid.

Typically, the doses of olaparib and abiraterone acetate are administered in the form of oral dosage forms such as capsules or tablets, and the initial doses are provided e.g. in two capsules or tablets (one containing abiraterone acetate and the other containing olaparib) and the follow-up dose is provided as one capsule or tablet containing olaparib and optionally a corticosteroid). Thus, a dosage regime normally involving as initial dose 2×150 mg olaparib, 4×250 mg abiraterone acetate plus 1×5 mg prednisone or prednisolone, i.e. 7 dosage forms) can be replaced with an initial dose of 1×300 mg olaparib+1×500 mg abiraterone acetate and optionally 1×5 mg prednisone or prednisone, i.e. 3 dosage forms and if olaparib and corticosteroid are contained in the same doses form, only 2 dosage forms are necessary for the initial dose. For the 12 hour dose, a reduction from 3 tablets to 2 or 1 tablets can be achieved. Thus, the traditional daily treatment involves 10 dosage forms, which can be reduced to 5 (if corticosteroid is in a separate dosage form) and preferably to 3 dosage forms.

The dosing regimen also includes the administration of a combination tablet that combines the co-amorphous form of Olaparib and Abiraterone acetate as a single tablet, optionally the combination tablet may further comprise a corticosteroid. The dosage regimen may include an initial dose of two combination tablets that comprise 100 or 150 mg Olaparib, 100-300 mg Abiraterone acetate and optionally 5 mg Prednisolone.

In the dosage regimen according to the invention, abiraterone acetate may be replaced by another prodrug of abiraterone, abiraterone (as free base) or enzalutamide. The initial and 12 hour dose is in a range of from 80 mg to 160 mg such as 80 mg or 160 mg.

### GENERAL

It should be understood that any feature and/or aspect discussed above in connection with the compounds according to the invention apply by analogy to the methods described herein.

The following figures and examples are provided below to illustrate the present invention. They are intended to be illustrative and are not to be construed as limiting in any way.

### BRIEF DESCRIPTION OF THE FIGURES

FIG. 1—X-ray diffractograms of the pure drug Olaparib as well as the co-amorphous Olaparib-BLG mixture at 50% drug loading, freshly prepared by ball milling (fresh) and after 15 months of storage at 40° C./75% RH.

FIG. 2—Powder dissolution of the co-amorphous Olaparib-BLG mixture at 50% drug loading prepared by ball milling in FaSSIF and 0.1 M HCl.

FIG. 3—mDSC thermogram of co-amorphous Olaparib-BLG mixture at 50% drug loading prepared by spray drying.

FIG. 4—mDSC thermogram of co-amorphous Olaparib-BLG mixture at 70% drug loading prepared by spray drying.

FIG. 5—X-ray diffractograms of co-amorphous Olaparib-BLG mixture at 50% drug loading prepared by spray drying.

FIG. 6—X-ray diffractograms of co-amorphous Olaparib-BLG mixture at 70% drug loading prepared by spray drying.

FIG. 7—SEM pictures of A) crystalline Olaparib, B) bulk BLG, C) Ball Mill Ola: BLG, 50:50, D) Spray dried Ola: BLG, 50:50, E) Spray dried Ola:BLG, 60:40, F) Spray dried Ola:BLG, 70:30, G) Spray dried Ola:Mannitol:BLG, 50:10:40, and H) Spray dried Ola:BLG, 50:50 (acetic acid).

FIG. 8—Powder dissolution in 0.1M HCl of the spray dried co-amorphous formulations Ola: BLG, 50:50, Ola:BLG, 60:40, Ola:BLG, 70:30, Ola:Mannitol:BLG, 50:10:40, Ola:BLG, 50:50 (acetic acid) as well as the powderized reference drug product Lynparza

FIG. 9—Powder dissolution in FaSSIF of the spray dried co-amorphous formulations Ola: BLG, 50:50, Ola:BLG, 60:40, Ola:BLG, 70:30, Ola:Mannitol:BLG, 50:10:40, Ola:BLG, 50:50 (acetic acid) as well as the powderized reference drug product Lynparza.

FIG. 10—Plasma-concentration profiles of Olaparib in Sprague Dawley rats after oral administration for the spray dried co-amorphous formulations Ola: BLG, 50:50, Ola:BLG, 60:40, Ola:BLG, 70:30, Ola:Mannitol:BLG, 50:10:40, Ola:BLG, 50:50 (acetic acid) as well as the powderized reference drug product Lynparza.

FIG. 11—Comparison of the AUC values determined from the plasma-concentration profiles of Olaparib in Sprague Dawley rats after oral administration for the spray dried co-amorphous

FIG. 12—X-ray diffractogram of co-amorphous Abiraterone acetate-BLG mixture at 30% drug loading prepared by spray drying.

FIG. 13—Powder dissolution in FeSSIF of the spray dried co-amorphous formulations Abiraterone acetate: BLG, 10:90; Abiraterone acetate: BLG, 20:80; Abiraterone acetate: BLG, 30:70; Abiraterone acetate: BLG, 60:40, as well as the powderized reference drug product Zytiga.

FIG. 14 Comparative pharmacokinetics of Co-amorphous form of Olaparib according to the invention vs Lynparza® in beagle dogs.

FIG. 15 X-ray diffractogram of co-amorphous Abiraterone acetate-BLG-Eudragit L mixture at 40% drug loading prepared by spray drying.

FIG. 16 m-DSC thermograms of co-amorphous Abiraterone acetate-BLG-Eudragit L mixture at 40% drug loading prepared by spray drying.

### EXAMPLES

### Materials

Two sources of Olaparib were used in these experiments: MSN, India and Olon, Italy.

Abiraterone acetate was obtained from Chemo Biosynthesis, Italy. Whey protein isolate (WPI), was obtained from Aria Food Ingredients.

All other materials and reagents were purchased from VWR, Thermo Fischer or Sigma Aldrich unless otherwise stated.

Beta-lactoglobulin was used with a purity of >98% in the protein fraction and was obtained from Aria Food Ingredients.

### Methods

### Ball Milling

One method of producing the co-amorphous forms was using vibrational ball milling (MixerMill MM400, Retsch GmbH & Co., Haan, Germany) in a 4° C. cold room for 60 min at 30 Hz. For this purpose, a total mass of 500 mg materials at the respective weight ratio between proteins and drug was weighed into 25 ml milling jar and milling was performed with two 12 mm stainless steel balls.

The other method of producing the co-amorphous forms was through spray drying.

Spray drying solution for olaparib:
The suitable amount of olaparib was dissolved in 80% ethanol (v/v). The concentration was 15 mg/ml.
Separately, protein solutions were prepared at 32, 50 and 75% mg/ml.
The final composition of the solvent mixture was 2:1 v/v (ethanol/water) except that one formulation incorporates an amount of acetic acid resulting in a solvent composition of ethanol/water/acetic acid at 65:30:5.
The final spray dried formulations were prepared by adding the protein solution into the olaparib solution under stirring. The ratio of protein solution and protein solution 1:5 (v/v) to achieve a final drug loading of 50%, 60% and 70% on solid basis.
Mannitol was included in one solid formulation resulting in a composition of olaparib/BLG/mannitol at a ratio of 50:40:10.

Spray drying solution for abiraterone acetate

Abiraterone acetate and BLG were dissolved in methanol (1.23-7.41 mg/ml) and acetic acid (100 mg/ml), separately. Prior to spray drying, BLG solution was added into abiraterone acetate solution at a volume ratio of 1 to 9, to achieve a 10%, 20%, 30% and 40% drug loading (i.e. 1:9, 2:8, 3:7 and 4:6 Abiraterone/BLG ratio) respectively.

Spray drying was carried out using a ProCepT spray drier (ProCepT, Zelzate, Belgium), equipped with a large cycloe and extended column using the following setup conditions:
Nozzle orifice: 0.6 mm
Inlet gas flow: 0.4 m³/min
Inlet temperature: 140° C.
Column out temperature: approx. 72° C.
Cyclone in temperature: approx. 55° C.
Cyclone gas flow: 3001/min
Dosing speed: approx. 5 g/min
Nozzle gas flow: 61/min

Secondary drying was performed under vacuum at 40° C. if required.

### X-Ray Powder Diffraction (XRPD) for Measurement of the Solid State Form

The presence of a fully amorphous formulation or one with crystallinity was measured using an X'Pert PANanalytical PRO X-ray diffractometer (PANanalytical, Almelo, The Netherlands) with Cu Kα radiation (λ=1.54187 Å). Samples were scanned in reflectance mode from 5° to 30° 2θ, with a scan speed of 0.067° 26/s and a step size of 0.026° 2θ. The acceleration voltage and current are 45 kV and 40 mA, respectively.

Modulate Temperature Differential Scanning Calorimetry (mDSC) for Measurement of the Glass Transition Temperature (Tg) and Homogeneity of the Co-Amorphous Forms

The mDSC thermograms of the samples were collected using a Discovery DSC (TA instruments, New Castle, USA) under a nitrogen gas flow of 50 ml/min. The samples were analysed at a heating rate of 2° C./min from −40° C. to 200° C., with an underlying modulation temperature amplitude of 0.2120° C. and a period of 40 s. A total of 6-8 mg sample powder was filled into aluminium Tzero pans and sealed with an aluminium Tzero lid. The glass transition temperature (Tg) was determined as the midpoint from the reversing heat flow signal.

### Scanning Electron Microscopy

The morphology of the selected amorphous powders was assessed by using a Hitachi TM3030 Tabletop scanning electron microscope (SEM, Hitachi High-Technologies Corporation, Tokyo, Japan). The samples were sputter-coated with a gold layer and imaged at an accelerating voltage of 15 kV.

### Powder Dissolution Testing in 0.1 M HCl, FaSSIF or FeSSIF

The powder dissolution of the samples was determined at room temperature in either 0.1 M HCl, fasted state simulated intestinal fluid V2 (FaSSIF V2—Biorelevant, London, UK), or fed state simulated intestinal fluid (FeSSIF V2—Biorelevant, London, UK) as dissolution medium. Samples equivalent to 100 mg (Olaparib) and 20 mg (Abiraterone) of drug were added into a 100 ml of Erlenmeyer flask containing 20 ml of dissolution medium. A magnetic stirring bar was added to the Erlenmeyer flask containing the dissolution medium and stirred at 200 rpm. At predetermined time points (5, 10, 20, 40, 60, 90, 120 min), 2 ml of dissolution medium were withdrawn from the dissolution vessels and immediately replaced by 2 ml of fresh dissolution medium. The dissolution samples were then filtered through a 0.45 μm filter and diluted using acetonitrile, and subsequently filtered again through a 0.45 μm filter.

The samples were analyzed toward drug content using high performance liquid chromatography (HPLC). For this purpose, an Agilent 1260 infinity HPLC system (Agilent, Santa Clara, USA) equipped with an Agilent 1290 Diode Array Detector was used.

### TGA Analysis

The moisture content of powders was measured using a Discovery Thermogravimetric Analyzer (TA Instruments, New Castle, USA). Sample was placed in a 100 μl platinum pan and heated from room temperature to 200° C. at a heating rate of 10° C./min. The weight loss in percent between room temperature and 140° C. was defined as the moisture content.

### Physical Stability

All samples were stored in a desiccator at 40° C. over a saturated sodium chloride solution to obtain 75% relative humidity. Samples were tested towards their solid state by XRPD at day 0 and subsequently at the timepoints reported.

### Preclinical Pharmacokinetic Evaluation

The pharmacokinetics of the selected formulations was evaluated using a rat model.

The animals all had pathogen free status and the housing and changing system was designed to assure that the pathogen free status had been preserved during the study. Trained personnel under veterinary supervision handled the animals. Health monitoring of the animal facilities was conducted according to standard operation procedures.

Six (6) groups with 5 Sprague Dawley rats (approximately 325 g in weight) in each group were administered with 6 different powder formulations of the test article, all were administered to animals at a dose level of 80 mg/kg. The co-amorphous formulations were reconstituted in MiliQ/demin water prior to administration via an oral syringe. At time-points (hrs) 0.5, 1.0, 2.0, 4.0, 6.0, 8.0 and 24, blood samples were drawn from each rat.

Approximately 200 μL blood was sampled from v. sublingualis per timepoint for plasma preparation and approximately 80 μL plasma was prepared for bioanalysis of test compound. Preparation of plasma for bioanalysis to determine test article concentration: The plasma was transferred to vials and stored frozen below −70° C. until analysis. Bioanalysis was carried out using LC-MS/MS method.

### Example 1—Co-Amorphous Forms of Olaparib/Beta-Lactoglobulin (BLG) Produced by Ball Milling

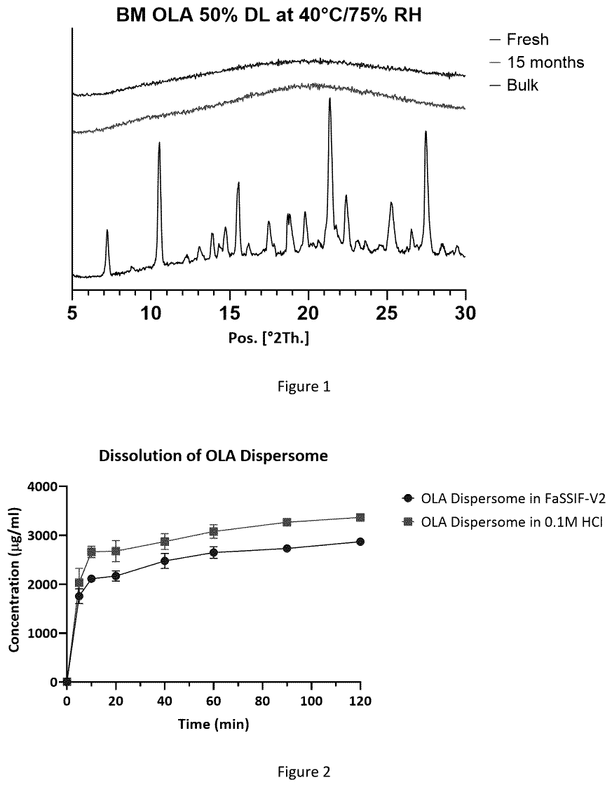

The ball milled Olaparib and BLG at 50% drug loading was confirmed to be fully amorphous using XRD. Indeed, the amorphous state was maintained after 15 months storage in the accelerated conditions (40° C., 75% RH), which confirms the significant stability afforded by the BLG even at a very high drug loading. The results are shown in FIG. 1.

The ball milled co-amorphous olaparib-BLG was then subjected to a dissolution evaluation at the gastric and FaSSIF conditions, respectively. The results have shown a rapid release from the ball milled powder under both conditions, with a slightly higher release in the gastric conditions comparing with the FaSSIF, perhaps a result of the lower pH. The results are shown in FIG. 2.

### Example 2—Co-Amorphous Forms of Olaparib/BLG Produced by Spray Drying

Spray drying is an alternative means of preparing an amorphous solid dispersion. The following formulations of Olaparib and BLG dispersions at different drug to BLG ratios were prepared via spray drying (note, one of the formulations also contained mannitol as the ratio shown in the table):

| Formulation | Solvent |
|---|---|
| Olaparib:BLG = 50:50 (w/w) | Ethanol/water mixture (2:1, v/v) |
| Olaparib:BLG = 60:40 (w/w) | Ethanol/water mixture (2:1, v/v) |
| Olaparib:BLG = 70:30 (w/w) | Ethanol/water mixture (2:1, v/v) |
| Olaparib:BLG:mannitol = 50:40:10 | Ethanol/water mixture (2:1, v/v) |
| (w/w/w) | |
| Olaparib:BLG = 50:50 with | Ethanol/water/acetic acid mixture |
| acetic acid (w/w) | (65:30:5, v/v/v) |

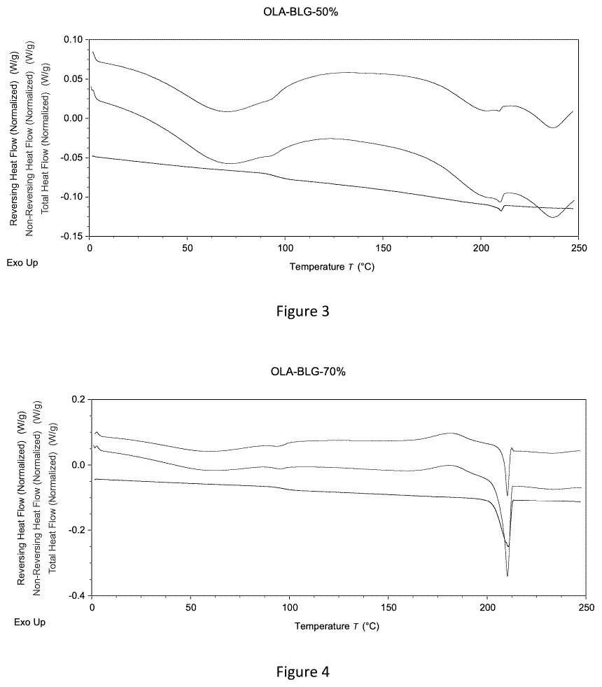

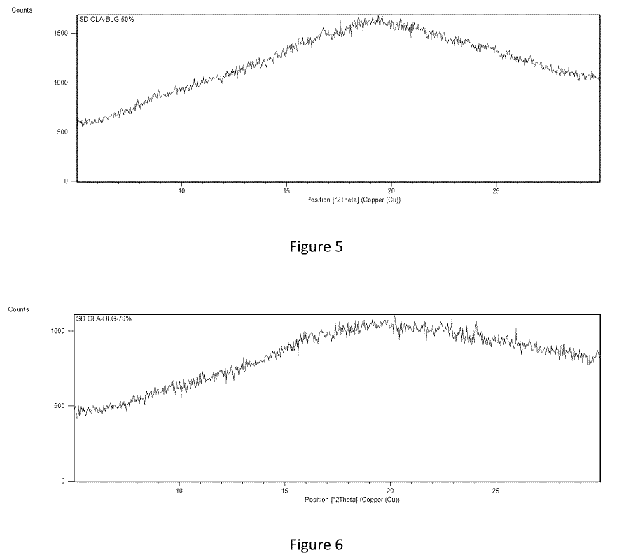

The physicochemical properties of the spray dried powders and their stability were evaluated over 11 weeks under stressed conditions (40° C., 75% RH). Examples of the XRD and DSC plots for the 50% and 70% drug loading are shown in the plots below (FIGS. 3-6):

The XRD data confirms that both formulations are amorphous, and the DSC results suggest that the glass transition occurs at greater than 90° C., thereby maintaining a high degree of drug stability. There is evidence of a melting event at about 210° C., which resulted from recrystallisation during sample heating above the glass transition during the mDSC run. The higher enthalpy at the higher drug loading is due to the higher drug content in the formulation. The recrystallisation would only occur at a temperature greater than the glass transition, which we would not expect any material would experience during storage and hence has no impact on drug product performance such as dissolution rates.

The data for all samples immediately after manufacturing and after 11 weeks storage is summarized in the Table below:

| | XRPD | | Tg (° C.) | | Moisture content | |
|---|---|---|---|---|---|---|
| Formulations | Fresh | 11 weeks | Fresh | 11 weeks | Fresh | 11 weeks |
| 50% | A* | A | 95.0 | 97.8 | 2.6 | 3.1 |
| 60% | A | A | 94.0 | 95.7 | 2.4 | 2.6 |
| 70% | A | A | 94.6 | 95.7 | 2.3 | 1.0 |
| 50% with mannitol | A | C** | 94.0 | N/A | 2.5 | 2.3 |
| 50% with Acetic acid | A | A | 94.0 | 98.7 | 6.7 | 3.7 |

*A denotes amorphous;
**C denotes crystalline (in this sample crystallinity is due to mannitol and not related to the drug Olaparib)

The data shows that the amorphous states of the spray dried powders were maintained during manufacture and “stressed” storage conditions. The crystalline peak seen with the sample containing mannitol was due to mannitol recrystallisation, rather than the results of recrystallisation of Olaparib.

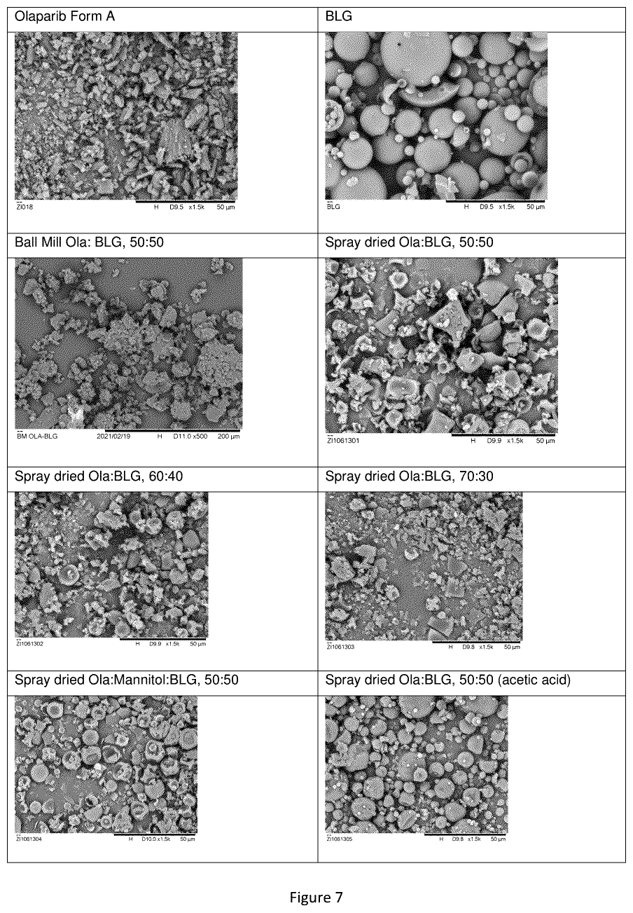

In addition, the microstructure of the spray dried powders was also analysed and the images are shown in FIG. 7; FIG. 7A is the first picture to the left, FIG. 7B is the first figure to the left etc.

It appears that there are a significant amount of angular shaped particles at the higher drug loading comparing with more globular shaped particles at the lower drug loading. It is possible that at higher drug loadings the morphological properties of the drug dominate the particle shape. The ball milled sample showed a different morphology in particle shape and size as a result of the different preparation method.

### Example 3—the In-Vitro Dissolution Profiles of the Spray Dried Olaparib-BLG Powder

The in-vitro dissolution of these spray dried co-amorphous powder formulations, alongside the reference drug product Lynparza (the tablet was first ground to a fine powder using a pestle and mortar). The dissolution for Olaparib was carried out at an equivalent of 5 mg/ml API for the samples. Lynparza was ground up and equivalent amount weighed out for testing as powder. The data shows a rapid dissolution of the co-amorphous formulations, with a relatively much higher dissolution than that of the Lynparza formulation.

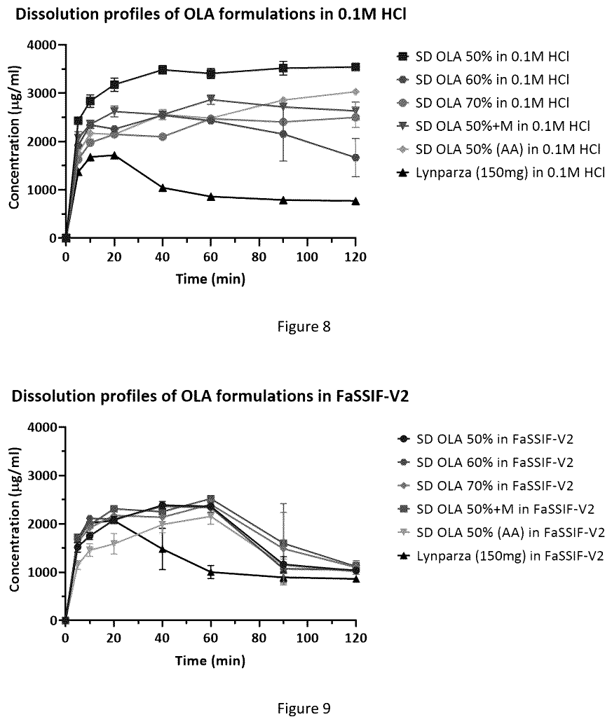

Under FaSSIF conditions, all of the spray dried powders had very similar dissolution profiles to each other, whereas under the gastric condition the 50% drug loading blend appeared to be faster. More importantly, despite of the lower overall polymer concentration in the spray dried powders when compared with the Reference (Lynparza contains a hot melt extruded Amorphous Solid Dispersion, with 30% drug and 70% copovidone (a synthetic polymer)), it is surprising that the BLG is able to sustain the drug supersaturation and minimize any drug precipitation during the course of the dissolution testing. This is relevant as a high supersaturation ratio allows the formation of a higher concentration gradient in the intestinal lumen that causes the flux across the absorption barrier and enhances drug absorption. Dissolution profiles are shown in FIGS. 8 and 9.

### Example 4—Rodent PK Evaluation of Spray Dried Olaparib Powders

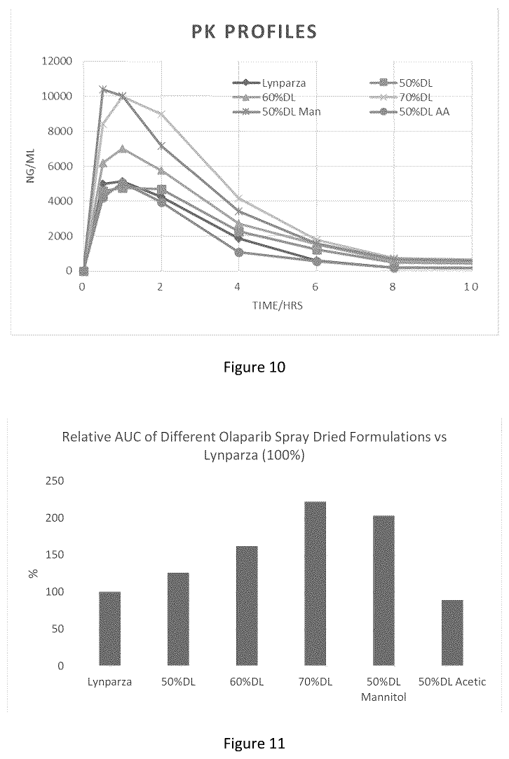

The spray dried co-amorphous formulations were subjected to PK evaluation using a rodent model. The PK profiles of the five test formulations alongside the Reference drug Lynparza are shown in FIG. 10.

The AUC (area under the curve) data is plotted with the Reference (Lynparza) normalized to 100%. Evidently that the 70% drug loading (DL), and the 50% drug loading incorporating mannitol exhibited the highest drug absorption. This is despite of the fact that there were no significant differences in the in-vitro drug dissolution profiles. It is very possible that higher supersaturation levels had been achieved in the animal GI tracts. The results are shown in FIG. 11.

### Example 5—Olaparib Tablet Formulations

At present, Lynparza is administered as 2 tablets at 150 mg dose strength, twice a day. The higher drug loading achieved by co-processing with BLG would allow the preparation of a single 300 mg tablet, or two smaller 150 mg tablets with a smaller size. This will have the benefit of reduced pill burden to the patients. The formulations below illustrate these possibilities.

Tablet 1 - 300 mg Olaparib

| Components | mg per Tablet |
|---|---|
| 70% DL co-amorphous Olaparib and BLG | 429 |
| Sorbitol | 171 |
| Sodium starch glycolate, Explotab ® | 48 |
| Sodium stearyl fumarate | 8 |
| Directly compressible starch, StarTab ® | 144 |
| Total | 800 |

Tablet 2 - 300 mg Olaparib

| Components | mg per Tablet |
|---|---|
| 70% DL co-amorphous Olaparib and BLG | 429 |
| Sorbitol | 71 |
| Sodium starch glycolate, Explotab ® | 48 |
| Sodium stearyl fumarate | 8 |
| Directly compressible starch, StarTab ® | 244 |
| Total | 800 |

Tablet 3 - 150 mg

| Components | mg per Tablet |
|---|---|
| 50% DL co-amorphous Olaparib and BLG | 300 |
| Sorbitol | 50 |
| Sodium starch glycolate, Explotab ® | 36 |
| Sodium stearyl fumarate | 6 |
| Directly compressible starch, StarTab ® | 208 |
| Total | 600 |

Tablet 4 -150 mg

| Components | mg per Tablet |
|---|---|
| 50% DL co-amorphous Olaparib | 300 |
| and BLG; and Mannitol | |
| Sorbitol | 50 |
| Sodium starch glycolate, Explotab ® | 36 |
| Sodium stearyl fumarate | 6 |
| Directly compressible starch, StarTab ® | 208 |
| Total | 600 |

### Example 6—Olaparib and Corticosteroid Tablet Formulations

These examples show that a corticosteroid can be combined with Olaparib and administered twice a day for the treatment of prostate cancers (mCRPC).

Tablet 1 - 300 mg Olaparib & 5 mg Prednisolone

| Components | mg per Tablet |
|---|---|
| 70% DL co-amorphous Olaparib and BLG | 429 |
| Prednisolone | 5 |
| Sorbitol | 171 |
| Sodium starch glycolate, Explotab ® | 48 |
| Sodium stearyl fumarate | 8 |
| Directly compressible starch, StarTab ® | 139 |
| Total | 800 |

Tablet 2 - 300 mg Olaparib & 5 mg Prednisone

| Components | mg per Tablet |
|---|---|
| 70% DL co-amorphous Olaparib and BLG | 429 |
| Prednisone | 5 |
| Sorbitol | 171 |
| Sodium starch glycolate, Explotab ® | 48 |
| Sodium stearyl fumarate | 8 |
| Directly compressible starch, StarTab ® | 139 |
| Total | 800 |

Tablet 3 - 300 mg Olaparib & 4 mg Methylprednisolone

| Components | mg per Tablet |
|---|---|
| 70% DL co-amorphous Olaparib and BLG | 429 |
| Methylprednisolone | 4 |
| Sorbitol | 171 |
| Sodium starch glycolate, Explotab ® | 48 |
| Sodium stearyl fumarate | 8 |
| Directly compressible starch, StarTab ® | 145 |
| Total | 805 |

Tablet 4 - 300 mg Olaparib & 0.5 mg Dexamethasone

| Components | mg per Tablet |
|---|---|
| 70% DL co-amorphous Olaparib and BLG | 429 |
| Dexamethasone | 0.5 |
| Sorbitol | 171 |
| Sodium starch glycolate, Explotab ® | 48 |
| Sodium stearyl fumarate | 8 |
| Directly compressible starch, StarTab ® | 148.5 |
| Total | 805 |

### Example 7—Co-Spray Drying of Abiraterone Acetate with BLG

The spray drying of the co-amorphous abiraterone and BLG at 10-40% drug loading was carried out as described previously.

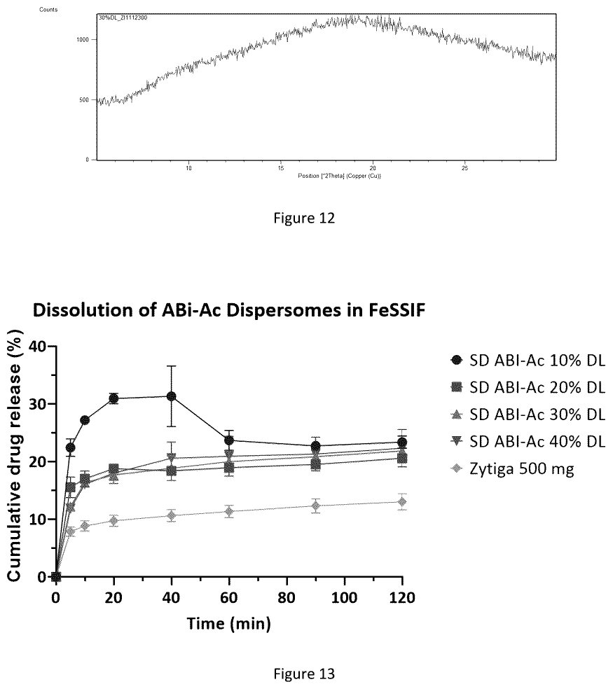

The formulations were characterized by XRD. The representative XRD diffractogram for the 30% DL spray dried powder is shown in FIG. 12.

This data shows that the freshly prepared spray dried powder is fully amorphous at 30% drug loading. The data for samples of other drug loading and storage over 5 weeks is summarized in the Table below.

XRPD

| Drug loading | Fresh | 5 weeks |
|---|---|---|
| 10% | A* | A |
| 20% | A | A |
| 30% | A | A |
| 40% | A | A |

*A = Amorphous

### Example 8—Dissolution Profiles of Co-Amorphous Abiraterone Acetate BLG Mixtures

The in-vitro performance of the spray dried co-amorphous abiraterone acetate BLG powders were assessed in the fed state simulated intestinal fluid. Abiraterone acetate is regarded as a drug product most affected by food effects among all FDA approved drug products. An in-vitro performance under the fed state is likely to be very relevant for its likely performance in vivo. This was evaluated alongside the Reference drug product Zytiga. The results are shown in FIG. 13.

The data shows that all of the Abiraterone acetate BLG spray dried powder formulations perform significantly better than the Zytiga tablet (ground to fine powder for this assessment). The 10% drug loading appeared to have a greater solubility at the initial stage of testing, which then falls to similar levels after one hour to the other formulations (20-40% DL).

### Example 9—Abiraterone Acetate Tablet Formulations

At present, abiraterone acetate is administered either as 4 tablets at 250 mg dose strength, or 2 tablets at 500 mg once a day. Zytiga 500 mg has a tablet weight in excess of 1.4 g, which frequently causes patient compliant issue because they are very large to be swallowed especially with a patient population that can be quite elderly.

The significantly increased solubility has the potential to be translated to higher bioavailability. As such a 250 mg tablet with a much smaller tablet size than the 500 mg tablet can be developed, thereby significantly increasing patient compliance.

Tablet 1 - 250 mg Abiraterone Acetate

| Components | mg per Tablet |
|---|---|
| 30% DL co-amorphous Abiraterone Acetate and BLG | 833 |
| Sorbitol | 67 |
| Sodium starch glycolate, Explotab ® | 60 |
| Sodium stearyl fumarate | 10 |
| Directly compressible starch, StarTab ® | 130 |
| Total | 1100 |

Tablet 2 - 250 mg Abiraterone Acetate

| Components | mg per Tablet |
|---|---|
| 40% DL co-amorphous Abiraterone Acetate and BLG | 625 |
| Sorbitol | 75 |
| Sodium starch glycolate, Explotab ® | 48 |
| Sodium stearyl fumarate | 8 |
| Directly compressible starch, StarTab ® | 144 |
| Total | 900 |

Tablet 3 - 250 mg Abiraterone Acetate

| Components | mg per Tablet |
|---|---|
| 30% DL co-amorphous Abiraterone Acetate and BLG | 833 |
| Sorbitol | 67 |
| Sodium starch glycolate, Explotab ® | 60 |
| Sodium stearyl fumarate | 5 |
| Sodium lauryl sulphate | 45 |
| Directly compressible starch, StarTab ® | 130 |
| Total | 1140 |

Tablet 4 - 250 mg Abiraterone Acetate

| Components | mg per Tablet |
|---|---|
| 40% DL co-amorphous Abiraterone Acetate and BLG | 625 |
| Sorbitol | 75 |
| Sodium starch glycolate, Explotab ® | 48 |
| Sodium stearyl fumarate | 5 |
| Sodium lauryl sulphate | 45 |
| Directly compressible starch, StarTab ® | 132 |
| Total | 930 |

### Example 10—Olaparib Formulations

### Capsule Formulation 100 mg (Test 3)

| Components | mg per Tablet |
|---|---|
| 70% DL co-amorphous Olaparib and BLG | 142.9 |
| Mannitol, Pearlitol 100 SD | 153.3 |
| Sodium starch glycolate, Explotab ® | 32.3 |
| Total | 298.5 |

The Olaparib-BLG co-amorphous (70% DL) formulation, mannitol and SSG were blended and filled into hard gelatin capsules size 0.

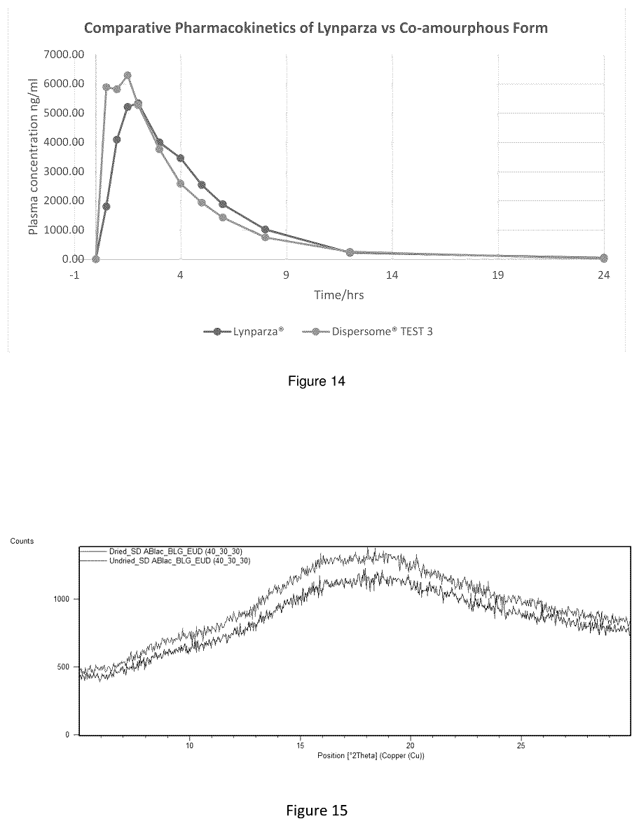

Formulation of Test 3 was used for a comparative pharmacokinetic evaluation versus the currently marketed tablet (Lynparza®). The pharmacokinetic profile in female beagle dogs is shown in FIG. 14, which shows a similar pharmacokinetic profile to Lynparza®. The co-amorphous formulation is at a 70% drug loading, whereas Lynparza® has about 30%. This shows that a much smaller dosage form (tablet or capsule) can be prepared via the co-amorphous formulation approach.

### Example 11—Olaparib and Corticosteroid Tablet Formulations

An experiment was carried out to determine whether Olaparib is chemically compatible with prednisolone, both alone and in the co-amorphous form.

No degradations of Olaparib or Prednisolone were observed when these materials were combined and after 1 month storage in sealed containers at 40° C. and 75% RH.

### Example 12—Ternary Co-Spray Drying of Abiraterone Acetate with BLG and Eudragit L100-55

As an alternative to the binary co-amorphous form of the Abiraterone and BLG, a ternary co-amorphous form of Abiraterone Acetate, BLG and Eudragit L100-55 was prepared.

### The Spray Drying Solution for a Ternary Formulation Comprising Abiraterone Acetate, BLG and Eudragit L100-55.

| Components | % w/W |
|---|---|
| Abiraterone Acetate | 3.41 |
| BLG | 2.56 |
| Eudragit L100-55 | 2.56 |
| Buthylated hyrdroxyanisol (BHA) | 0.007 |
| Buthylhydroxytoluene (BHT) | 0.007 |
| Acetic acid | 4.48 |
| 1-Propanol | 44.49 |
| Water | 42.49 |
| Total | 100.00 |

On completion of the spray drying, the spray dried powder has the following composition: Abiraterone Acetate: BLG: Eudragit L100-55, 40:30:30 with a small amount of antioxidants, BHA and BHT. The powder was subjected to vacuum drying. The XRPD profiles of the powder prior to and after vacuum drying is shown in FIG. 15, which demonstrates that the material is amorphous.

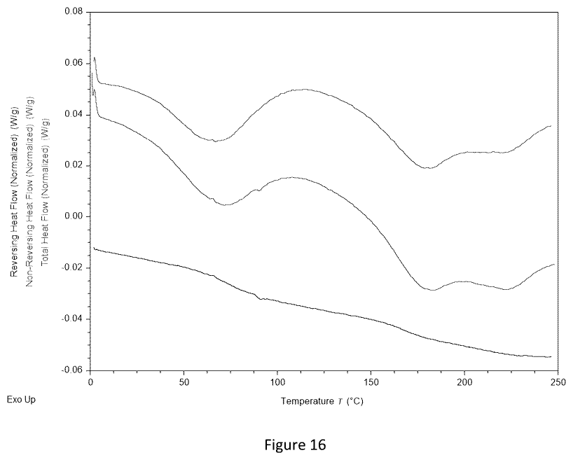

The DSC data (FIG. 16) show a major glass transition at around 73° C. and a second glass transition at around 167° C.

### Example 13—Abiraterone Acetate Tablet Formulations

Abiraterone acetate tablet formulations incorporating binary co-amorphous form (25% drug loading), and ternary co-amorphous form (40% and 50% drug loading respectively) were prepared. Crystalline drugs were also added to some of the formulations. The final dose strength of the tablets was 250 mg, except for the 100% Ternary formulation at 40% drug loading that had a dose strength of 125 mg.

TABLE t
formulations (% w/w)

| | | 100% co- | Mixed | 100% co- |
|---|---|---|---|---|
| | Mixed | amorphous | (Ternary | amorphous |
| | (Binary + 50% | (Ternary | 40% DL + 70% | (Ternary |
| | crystalline) | 50% DL) | Crystalline) | 40% DL) |
| Dose strength | 250 mg | 250 mg | 250 mg | 125 mg |
| Abiraterone acetate | 11.25 | 0.00 | 24.41 | 0.00 |
| (Crystalline) | | | | |
| ABA BLG 25:75 | 45.00 | 0.00 | 0.00 | 0.00 |
| ABA BLG Eudragit | 0.00 | 45.00 | 0.00 | 0.00 |
| 50:25:25 | | | | |
| ABA BLG Eudragit | 0.00 | 0.00 | 26.15 | 44.96 |
| 40:30:30 | | | | |
| Sorbitol, Parteck | 20.50 | 31.75 | 26.20 | 31.80 |
| SI 150 | | | | |
| Sodium starch | 15.00 | 15.00 | 15.00 | 14.99 |
| glycolate, Explotab | | | | |
| Colloidal silicon | 5.00 | 5.00 | 5.00 | 5.00 |
| dioxide, Aerosil | | | | |
| Pharma 200 | | | | |
| Sodium lauryl | 2.25 | 2.25 | 2.25 | 2.25 |
| sulphate, Sigma | | | | |
| Aldrich's Emprove | | | | |
| Essential | | | | |
| Magnesium stearate, | 1.00 | 1.00 | 1.00 | 1.00 |
| Ligamed MF-2-V | | | | |

These formulations were used for a preclinical evaluation in male beagle dogs. Two reference drugs were tested alongside these formulations, Zytiga® 500 mg and Yonsa® 125 mg (×2). 250 mg Yonsa® tablets have been shown to be bioequivalent to 500 mg Zytiga® in human clinical studies.

The pharmacokinetic summary results are shown in the table below.

| | | | Mixed | | | |
|---|---|---|---|---|---|---|
| | Mixed | 100% co- | (Tern | 100% co- | | |
| | (Binary + | amorphous | 40% DL + | amorphous | | |
| | 50% | (Tern 50% | 70% | (Tern | Yonsa | Zytiga |
| | crystalline) | DL) | Crystalline) | 40% DL) | 125 mg × 2 | 500 mg |
| Administered | 250 mg | 250 mg | 250 mg | 125 mg | 250 mg | 500 mg |
| dose | | | | | | |
| Cmax | 387 | 343 | 336 | 572 | 203 | 171 |
| Cmax, % CV | 31 | 34 | 48 | 31 | 47 | 79 |
| AUC (0-t) | 580 | 789 | 691 | 997 | 423 | 330 |
| AUC (0-t), % | 30 | 35 | 89 | 40 | 62 | 73 |
| CV | | | | | | |
| AUC (0-inf) | 607 | 846 | 776 | 1040 | 446 | 426 |
| AUC (0-inf), | 31 | 35 | 92 | 40 | 57 | 56 |
| % CV | | | | | | |
| Tmax | 1 | 1 | 1.5 | 1 | 1.5 | 1 |
| T½ | 2.61 | 3.27 | 2.68 | 2.96 | 3.15 | 2.87 |

These data conclusively demonstrated that the co-amorphous formulations provide a substantial increase in the bioavailability of Abiraterone acetate. The (100%) ternary co-amorphous formulation at half the dose of Yonsa, and a quarter dose of Zytiga still shows a substantially higher bioavailability than these formulations.

### Example 14—Abiraterone Acetate Tablet Formulations

Tablet 1 - 70 mg Abiraterone Acetate

| Components | mg per Tablet |
|---|---|
| 40% DL co-amorphous Abiraterone Acetate, | 175 |
| BLG and Eudragit | |
| Functional Calcium Carbonate | 37.19 |
| Sodium starch glycolate, Explotab ® | 4.38 |
| Magnesium stearate | 2.19 |
| Total | 218.75 |

Tablet 2 - 75 mg Abiraterone Acetate

| Components | mg per Tablet |
|---|---|
| 40% DL co-amorphous Abiraterone Acetate, | 187.50 |
| BLG and Eudragit | |
| Functional Calcium Carbonate | 72.32 |
| Sodium starch glycolate, Explotab ® | 5.36 |
| Magnesium stearate | 2.68 |
| Total | 267.86 |

Tablet 3 - 250 mg Abiraterone Acetate

| Components | mg per Tablet |
|---|---|
| 40% DL co-amorphous Abiraterone Acetate, | 562.50 |
| BLG and Eudragit | |
| Functional Calcium Carbonate | 216.96 |
| Sodium starch glycolate, Explotab ® | 16.08 |
| Magnesium stearate | 8.04 |
| Total | 803.58 |

### Example 15—Abiraterone Acetate and Olaparib Tablet Formulations

Tablet 1 A combination tablet comprising 125mg
Abiraterone Acetate, 150mg Olaparib and 5mg

| Components | mg per Tablet |
|---|---|
| 40% DL co-amorphous Abiraterone Acetate, | 281.25 |
| BLG and Eudragit | |
| 70% DL co-amorphous Olaparib and BLG | 214.28 |
| Prednisolone | 5 |
| Functional Calcium Carbonate | 216.96 |
| Sodium starch glycolate, Explotab ® | 16.08 |
| Magnesium stearate | 8.04 |
| Total | 741.61 |

Tablet 2 A combination tablet comprising 125 mg
Abiraterone Acetate, 150 mg Olaparib and 5 mg

| Components | mg per Tablet |
|---|---|
| 40% DL co-amorphous Abiraterone Acetate, | 281.25 |
| BLG and Eudragit | |
| 70% DL co-amorphous Olaparib and BLG | 214.28 |
| Prednisolone | 5 |
| Functional Calcium Carbonate | 108.48 |
| Mannitol | 108.48 |
| Sodium starch glycolate, Explotab ® | 16.08 |
| Magnesium stearate | 8.04 |
| Total | 741.61 |

Tablet 3 A combination tablet comprising 200mg
Abiraterone Acetate, 150mg Olaparib and 5mg

| Components | mg per Tablet |
|---|---|
| 40% DL co-amorphous Abiraterone Acetate, | 500.00 |
| BLG and Eudragit | |
| 70% DL co-amorphous Olaparib and BLG | 214.28 |
| Prednisolone | 5 |
| Functional Calcium Carbonate | 108.48 |
| Mannitol | 108.48 |
| Sodium starch glycolate, Explotab ® | 16.08 |
| Magnesium stearate | 8.04 |
| Total | 960.36 |

### Example 16—Abiraterone Acetate and Niraparib Tablet Formulations

Tablet 1 A combination tablet comprising 125mg
Abiraterone Acetate, 100mg Niraparib and 5mg

| Components | mg per Tablet |
|---|---|
| 40% DL co-amorphous Abiraterone Acetate, | 281.25 |
| BLG and Eudragit | |
| Niraparib | 100 |
| Prednisolone | 5 |
| Functional Calcium Carbonate | 216.96 |
| Sodium starch glycolate, Explotab ® | 16.08 |
| Magnesium stearate | 8.04 |
| Total | 627.33 |

Tablet 2 A combination tablet comprising 250mg
Abiraterone Acetate, 200mg Niraparib and 10mg

| Components | mg per Tablet |
|---|---|
| 40% DL co-amorphous Abiraterone Acetate, | 562.50 |
| BLG and Eudragit | |
| Niraparib | 200 |
| Prednisolone | 10 |
| Functional Calcium Carbonate | 320.00 |
| Sodium starch glycolate, Explotab ® | 32.16 |
| Magnesium stearate | 16.08 |
| Total | 1140.74 |

1. A co-amorphous form of beta-lactoglobulin and a drug substance, wherein the drug substance is selected from olaparib or abiraterone acetate and, wherein the concentration of the drug substance in the co-amorphous form is from 10% to 90% w/w based on the total weight of the co-amorphous form.

2-34. (canceled)

35. The co-amorphous form according to claim 1, wherein the drug substance is olaparib and the concentration of olaparib in the co-amorphous form is from 20% to 90% w/w based on the total weight of the co-amorphous form.

36. The co-amorphous form according to claim 1, wherein the drug substance is abiraterone acetate and the concentration of abiraterone acetate in the co-amorphous form is from 10% to 70% w/w based on the total weight of the co-amorphous form.

37. The co-amorphous form according to claim 1, wherein the purity of beta-lactoglobulin is 92% or more.

38. The co-amorphous from according to claim 1, wherein the co-amorphous form is admixed with a co-amorphous form of beta-lactoglobulin and a corticosteroid.

39. The co-amorphous form according to claim 1, wherein the co-amorphous form is a co-amorphous form of beta-lactoglobulin, a corticosteroid and the drug substance selected from olaparib or abiraterone acetate.

40. The co-amorphous form according to claim 1, further comprising niraparib.

41. A composition comprising the co-amorphous form of claim 1.

42. The composition according to claim 41, further comprising a pharmaceutically acceptable excipient.

43. The composition according to claim 41, wherein the composition comprises from 5% to 100% w/w of the co-amorphous form, based on the total weight of the composition.

44. The composition according to claim 41, wherein the drug substance is olaparib and the concentration of the co-amorphous form is from 30% to 80% w/w of the total weight of the composition.

45. The composition according to claim 41, wherein the drug substance is abiraterone acetate and the concentration of the co-amorphous form is from 40% to 85% w/w of the total weight of the composition.

46. The composition according to claim 41, further comprising one or more corticosteroids.

47. The composition according to claim 41, wherein the composition is formulated for oral administration.

48. The composition according to claim 41, wherein the composition is a solid dosage form.

49. The composition according to claim 41, further comprising niraparib.

50. A dosage regime comprising daily administration of two tablets, wherein each tablet comprises 100-150 mg olaparib and 100-150 mg abiraterone acetate as a single fixed combination, twice a day and, wherein the dose of olaparib and abiraterone acetate is contained in a co-amorphous form as defined in claim 1.

51. The dosage regime according to claim 50, wherein the single fixed combination in each tablet further comprises prednisone or prednisolone.
